(19) United States
(12) Patent Application Publication
(10) Pub. No.: US 2026/0061146 A1
(43) Pub. Date: Mar. 5, 2026

(54) SYSTEMS AND METHODS FOR PULMONARY FUNCTION TESTING ON RESPIRATORY THERAPY DEVICES

(71) Applicant: ResMed Digital Health Inc., San Diego, CA (US)

(72) Inventors: Genevieve Claire ATKINSON, Sydney (AU); Liam HOLLEY, Sydney (AU); Gregory Robert PEAKE, Sydney (AU); Andrew William GILLETT, Sydney (AU); Sakeena DE SOUZA, Sydney (AU)

(21) Appl. No.: 19/106,932

(22) PCT Filed: Aug. 24, 2023
(86) PCT No.: PCT/US2023/031091

§ 371 Date: Feb. 26, 2025

Related U.S. Application Data
(60) Provisional application No. 63/373,959, filed on Aug. 30, 2022.

(51) Int. Cl. A61M 16/00; A61M 16/06

(52) U.S. Cl. CPC A61M 16/024; A61M 16/06; A61M 2016/0039; A61M 2205/502

(57) ABSTRACT

A method for testing lung function of a user donning a user interface includes administering a supplemental breathing test in response to receiving an input from the user selecting the supplemental breathing test. The user interface is coupled, via a conduit, to a respiratory therapy device in a respiratory therapy system. Moreover, the supplemental breathing test is administered by adjusting parameters of the respiratory therapy device. The method also includes receiving first airflow parameter data associated with a first airflow generated by the user. The first airflow is generated by the user into an interior of the user interface while donning the user interface. The method also includes characterizing the lung function of the user based at least in part on the first airflow parameter data.

### CROSS-REFERENCE TO RELATED APPLICATIONS

This application claims the benefit of, and priority to, U.S. Provisional Ser. No. 63/373,959 filed on Aug. 30, 2022, which is hereby incorporated by reference herein in its entirety.

### TECHNICAL FIELD

The present disclosure relates generally to systems and methods for respiratory therapy devices, and more particularly, to systems and methods for testing pulmonary function of individuals using respiratory therapy devices.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. However, some users find such systems to be uncomfortable, difficult to use, expensive, aesthetically unappealing and/or fail to perceive the benefits associated with using the system. As a result, some users will elect not to use the respiratory therapy system or discontinue use of the respiratory therapy system absent a demonstration of the severity of their symptoms when respiratory therapy treatment is not used or encouragement or affirmation that the respiratory therapy system is improving their sleep quality and reducing the symptoms of these disorders. The present disclosure is directed to solving these and other problems.

### SUMMARY

According to some implementations of the present disclosure, a method for testing lung function of a user donning a user interface includes administering a supplemental breathing test in response to receiving an input from the user selecting the supplemental breathing test. The user interface is coupled, via a conduit, to a respiratory therapy device in a respiratory therapy system. Moreover, the supplemental breathing test is administered by adjusting parameters of the respiratory therapy device. The method also includes receiving first airflow parameter data associated with a first airflow generated by the user. The first airflow is generated by the user into an interior of the user interface while donning the user interface. The method also includes characterizing the lung function of the user based at least in part on the first airflow parameter data.

According to some implementations of the present disclosure, a respiratory therapy system includes a respiratory therapy device, a conduit, and a user interface. The user interface is coupled to the respiratory therapy device via the conduit. The system also includes a memory device, and a control system. The memory stores machine-readable instructions. The control system includes one or more processors configured to execute the machine-readable instructions to administer a supplemental breathing test in response to receiving an input from the user selecting the supplemental breathing test. The supplemental breathing test is administered by adjusting parameters of the respiratory therapy device. The control system is further configured to receive first airflow parameter data associated with a first airflow generated by the user. The first airflow is generated by the user into an interior of the user interface while donning the user interface. The control system is further configured to characterize the lung function of the user based at least in part on the first airflow parameter data.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2A is a perspective view of at least a portion of the system of FIG. 1, a user, and a bed partner, according to some implementations of the present disclosure;

FIG. 2B is a perspective view of at least a portion of the system of FIG. 1, a user testing lung function, according to some implementations of the present disclosure;

FIG. 3A is a perspective view of a user interface, according to some implementations of the present disclosure;

FIG. 3B is an exploded view of the user interface of FIG. 3A, according to some implementations of the present disclosure;

FIG. 4 illustrates an exemplary timeline for a sleep session, according to some implementations of the present disclosure;

FIG. 5 illustrates an exemplary hypnogram associated with the sleep session of FIG. 4, according to some implementations of the present disclosure;

FIG. 6A is a process flow diagram for a method for testing lung function of a user donning a user interface of a respiratory therapy system, according to some implementations of the present disclosure;

FIG. 6B is a process flow diagram for a method for ensuring the user is ready for the supplemental breathing test to begin, according to some implementations of the present disclosure;

FIG. 6C is a process flow diagram for a method for monitoring a user's lung function, according to some implementations of the present disclosure; and

FIG. 6D is a process flow diagram for a method for characterizing the lung function of a user, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

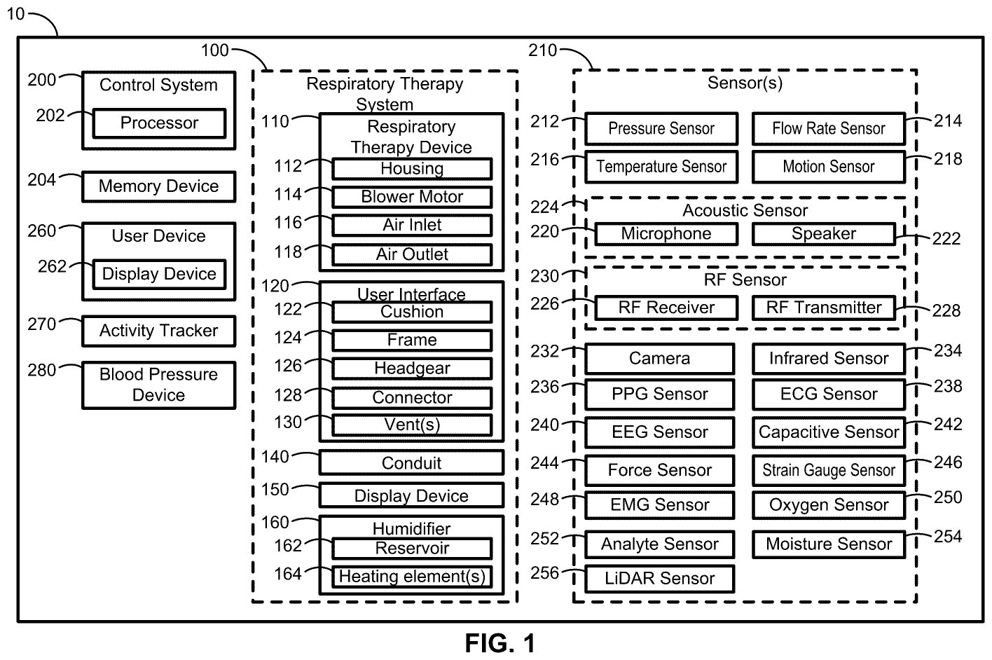

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

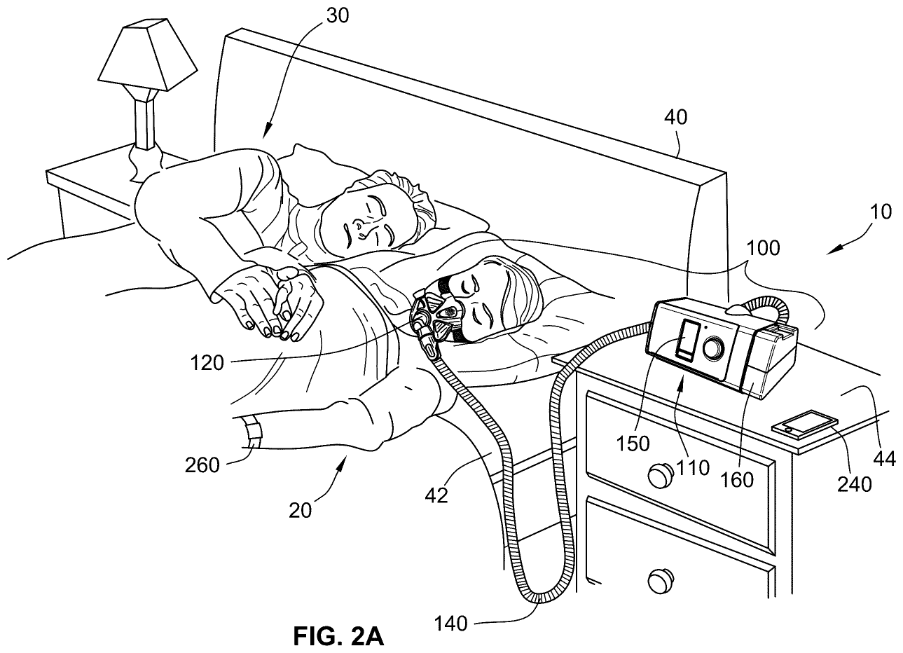

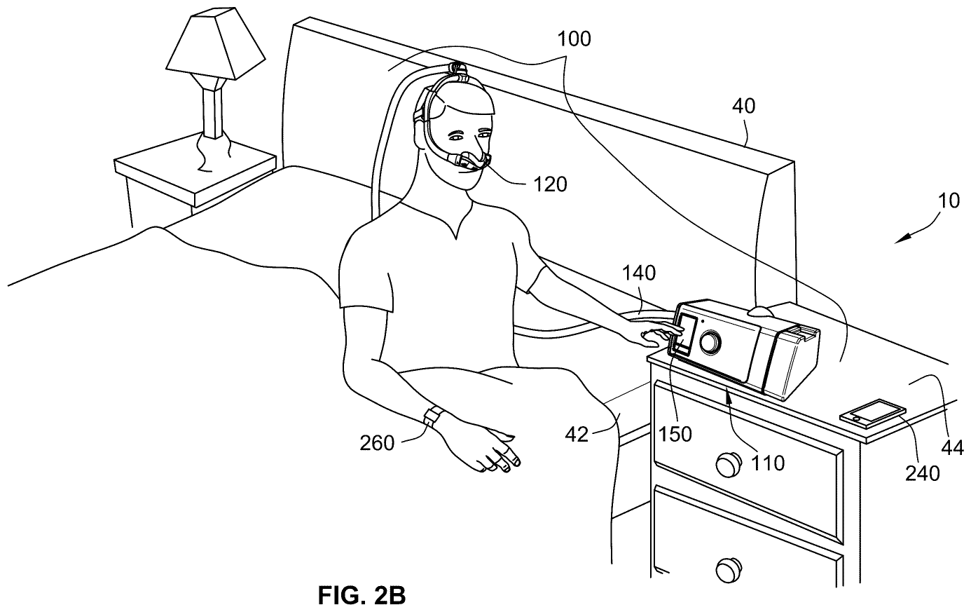

As shown in FIG. 2A, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2A, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

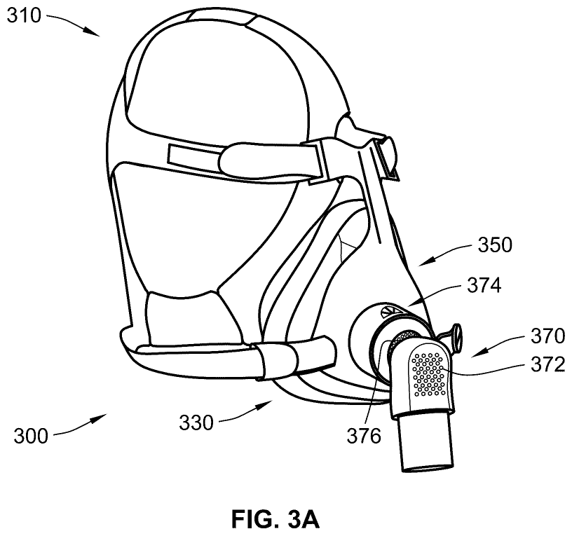

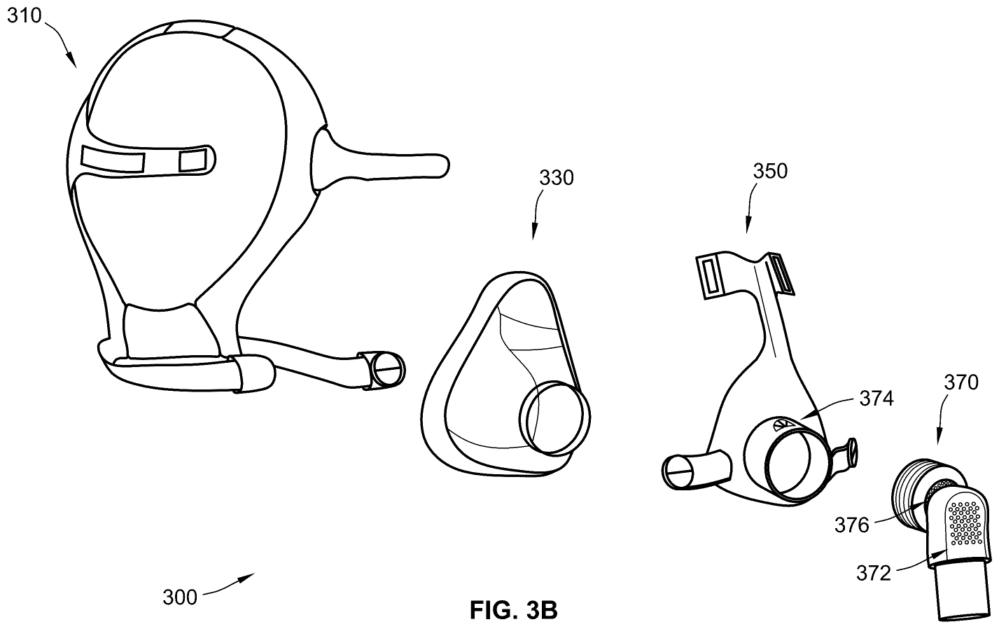

The respiratory therapy device 110 includes a housing 112, a blower motor 114, an air inlet 116, and an air outlet 118 (FIG. 1). Referring to FIGS. 3A and 3B, the blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). As shown in FIGS. 3A and 3B, the housing 112 can include a vent 113 to allow air to pass through the housing 112 to the air inlet 116. As described below, the conduit 140 is coupled to the air outlet 118 of the respiratory therapy device 110.

Referring back to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2A, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

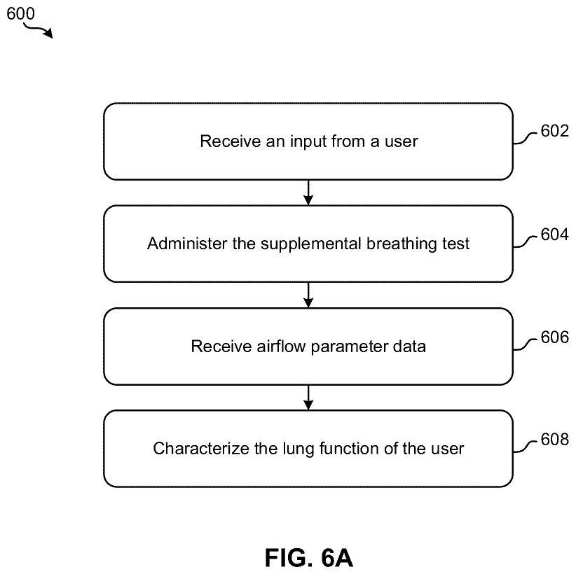

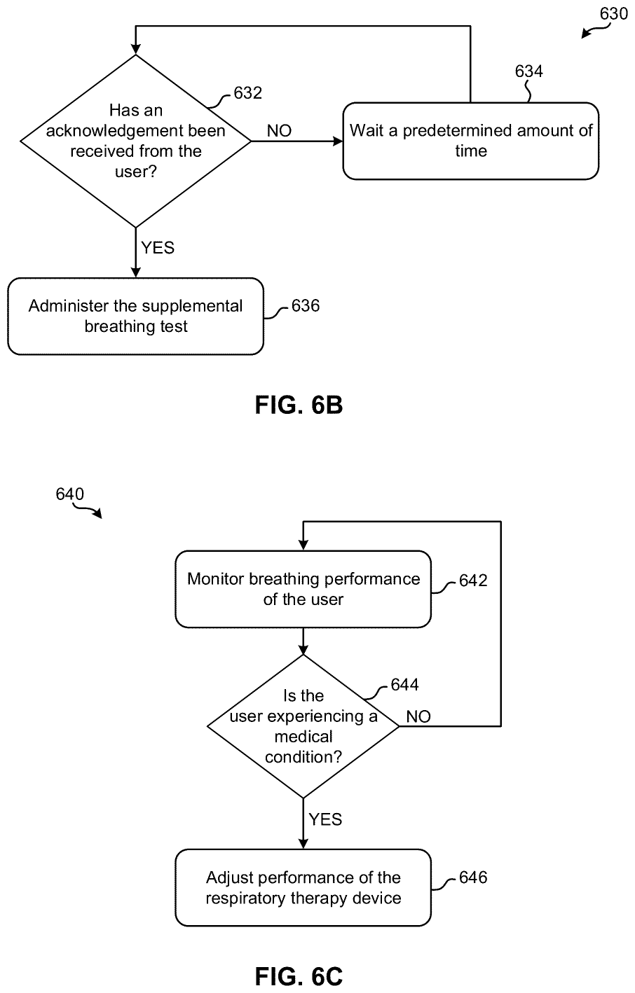

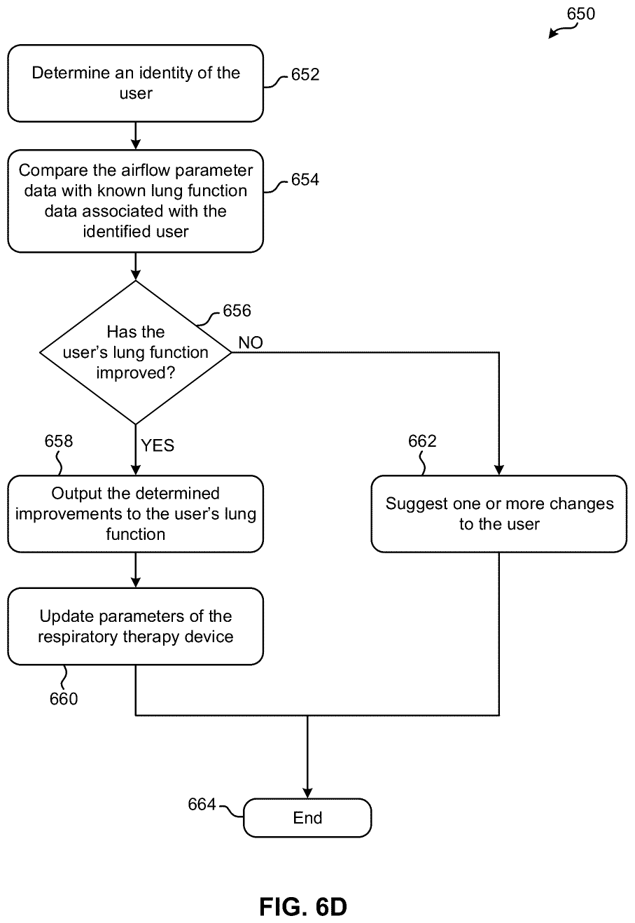

While FIG. 2A depicts the user 20 donning the user interface 120 while lying in bed (e.g., during a sleep session), in some situations the user 20 may don the user interface 120 before lying down in the bed. For instance, FIG. 2B shows the user 20 sitting on the bed 40 while donning the user interface 120. The user may thereby select one or more supplemental functions that are supported by the respiratory therapy system. According to an example, which is in no way intended to limit the invention, the user may select a supplemental breathing test and/or exercise designed to improve the user's lung function, e.g., as will be described in further detail below (e.g., see FIGS. 6A-6D).

Referring now to FIGS. 3A and 3B, a user interface 300 that is the same as, or similar to, the user interface 120 (FIG. 1) according to some implementations of the present disclosure is illustrated. The user interface 300 generally includes a cushion 330 and a frame 350 that define a volume of space around the mouth and/or nose of the user. When in use, the volume of space receives pressurized air for passage into the user's airways. In some implementations, the cushion 330 and frame 350 of the user interface 300 form a unitary component of the user interface. The user interface 300 can also include a headgear 310, which generally includes a strap assembly and optionally a connector 370. The headgear 310 is configured to be positioned generally about at least a portion of a user's head when the user wears the user interface 300. The headgear 310 can be coupled to the frame 350 and positioned on the user's head such that the user's head is positioned between the headgear 310 and the frame 350. The cushion 330 is positioned between the user's face and the frame 350 to form a seal on the user's face. The optional connector 370 is configured to couple to the frame 350 and/or cushion 330 at one end and to a conduit of a respiratory therapy device (not shown). The pressurized air can flow directly from the conduit of the respiratory therapy system into the volume of space defined by the cushion 330 (or cushion 330 and frame 350) of the user interface 300 through the connector 370). From the user interface 300, the pressurized air reaches the user's airway through the user's mouth, nose, or both. Alternatively, where the user interface 300 does not include the connector 370, the conduit of the respiratory therapy system can connect directly to the cushion 330 and/or the frame 350.

In some implementations, the connector 370 may include one or more vents 372 (e.g., a plurality of vents) located on the main body of the connector 370 itself and/or one or a plurality of vents 376 (“diffuser vents”) in proximity to the frame 350, for permitting the escape of carbon dioxide (CO₂) and other gases exhaled by the user. In some implementations, one or a plurality of vents, such as vents 372 and/or 376 may be located in the user interface 300, such as in frame 350, and/or in the conduit 140. In some implementations, the frame 350 includes at least one anti-asphyxia valve (AAV) 374, which allows CO₂ and other gases exhaled by the user to escape in the event that the vents (e.g., the vents 372 or 376) fail when the respiratory therapy device is active. In general, AAVs (e.g., the AAV 374) are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 376, other masks might have only the plurality of vents 372 on the connector itself).

As noted above, the specific details shown in FIGS. 2A-3B are in no way intended to be limiting. Rather, other implementations may include user interfaces that are the same as, similar to, or different from the user interfaces 120 depicted in FIGS. 2A-3B. For instance, some user interfaces are indirect user interfaces, while other user interfaces are direct user interfaces. An indirectly connected user interface may deliver pressurized air from the conduit 140 of the respiratory therapy system to the cushion and/or frame through a user interface conduit, rather than directly from the conduit 140 of the respiratory therapy system.

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

In some implementations, the conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a my Air™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air may flow from an air inlet through a blower motor, and then through a humidifier before exiting the respiratory therapy device 110 via air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to back to FIG. 1, the one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIGS. 2A-2B) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIGS. 2A-2B), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones The speaker 222 outputs sound waves that are audible to a user of the system 10 (e.g., the user 20 of FIGS. 2A-2B). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIGS. 2A-2B). Based at least in part on the data from the microphone 220 and/or the speaker 222, the control system 200 can determine a location of the user 20 (FIGS. 2A-2B) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIGS. 2A-2B), to determine air flow of the mouth and/or nose of the user, to determine a time when the user enters the bed (FIGS. 2A-2B), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIGS. 2A-2B) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIGS. 2A-2B, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIGS. 2A-2B, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

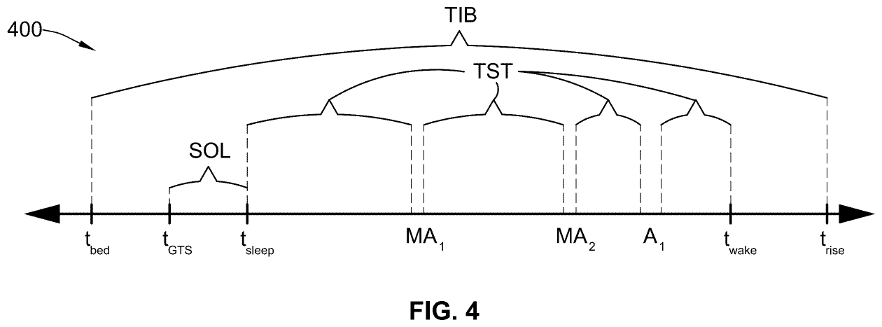

Referring to the timeline 400 in FIG. 4 the enter bed time t_{bed} is associated with the time that the user initially enters the bed (e.g., bed 40 in FIGS. 2A-2B) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 400 of FIG. 4, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

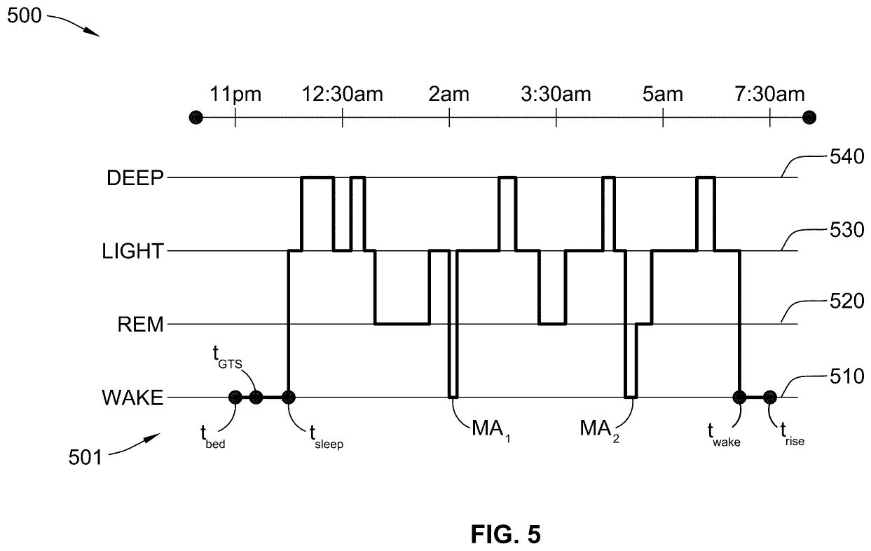

Referring to FIG. 5, an exemplary hypnogram 500 corresponding to the timeline 400 (FIG. 4), according to some implementations, is illustrated. As shown, the hypnogram 500 includes a sleep-wake signal 501, a wakefulness stage axis 510, a REM stage axis 520, a light sleep stage axis 530, and a deep sleep stage axis 540. The intersection between the sleep-wake signal 501 and one of the axes 510-540 is indicative of the sleep stage at any given time during the sleep session.

The sleep-wake signal 501 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 500 is shown in FIG. 5 as including the light sleep stage axis 530 and the deep sleep stage axis 540, in some implementations, the hypnogram 500 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 500 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

As previously mentioned, respiratory therapy systems may be used to treat disorders such as those that affect the quality of sleep experienced by an individual. Consistent and continued use of respiratory therapy systems, e.g., such as those included herein, are able to not only improve the quality of sleep experienced by an individual, but also the overall lung function of the individual. By regularly conditioning a user's respiratory system (e.g., lungs), the user may be able to improve a number of their respiratory-related metrics. For example, a user that consistently uses a PAP system (e.g., see 100 of FIG. 1) during their sleep sessions may benefit from increased lung capacity over time. It follows that various ones of the respiratory therapy systems included herein are able to achieve a number of improvements for users.

While the benefits of using the various respiratory therapy systems included herein are substantial, these benefits may not always be readily apparent to an individual that uses a respiratory therapy system. For example, improvements to the quality and/or amount of sleep experienced by a user during a given sleep session may be noticeable to some users after the user awakens. However, improvements to a user's lung capacity and general lung function may not be noticeable to the user under normal conditions. As a result, some users may not fully appreciate the benefits associated with continued use of a respiratory therapy system during sleep sessions. These users are less likely to continue using respiratory therapy systems, thereby ultimately decreasing lung function and reducing the quality of sleep experienced by users during future sleep sessions.

In sharp contrast to these conventional shortcomings, various ones of the implementations included herein are able to provide information to users that quantifies the various improvements to the user's respiratory system. As a result, users are able to understand the various benefits that are achieved as a result of utilizing respiratory therapy systems, even if the improvements are not readily noticeable to the user. This can act as a positive feedback loop, encouraging users to continue using respiratory therapy systems during sleep sessions, and ultimately improving user experience.

Referring to FIG. 6A, a method 600 for testing lung function of a user donning a user interface of a respiratory therapy system according to some implementations of the present disclosure is illustrated. By causing the respiratory therapy device to administer breathing related exercises and tests for a user, method 600 is able to determine improvements to lung function of the user. While the configuration of the respiratory therapy system may differ depending on the implementation, method 600 has been presented below in the context of a system having a user interface that is coupled to a respiratory therapy device via a conduit (e.g., see FIGS. 2A-2B). It follows that one or more steps of the method 600 can be implemented using any element or aspect of the system 100 (FIGS. 1-2B) described herein.

More or less operations than those specifically described in FIG. 6A may be included in method 600, as would be understood by one of skill in the art upon reading the present descriptions. Each of the steps of the method 600 may be performed by any suitable component of the operating environment. For example, in various implementations, the method 600 may be partially or entirely performed by a controller, a processor, a computer, etc., or some other device having one or more processors therein. Thus, in some implementations, method 600 may be a computer-implemented method. Moreover, the terms computer, processor and controller may be used interchangeably with regards to any of the implementations herein, such components being considered equivalents in the many various permutations of the present invention.

Moreover, for those implementations having a processor, the processor, e.g., processing circuit(s), chip(s), and/or module(s) implemented in hardware and/or software, and preferably having at least one hardware component may be utilized in any device to perform one or more steps of the method 600. Illustrative processors include, but are not limited to, a central processing unit (CPU), an application specific integrated circuit (ASIC), a field programmable gate array (FPGA), etc., combinations thereof, or any other suitable computing device known in the art.

As shown in FIG. 6A, operation 602 of method 600 includes receiving an input from a user. The user selects a supplemental breathing test that is of interest to the user and provides an input indicating that the supplemental breathing test has been selected. With respect to the present description, use of the term “supplemental breathing test” is in no way intended to limit the invention. Rather, a supplemental breathing test is intended to refer to any breathing related process that may be administered by a respiratory therapy system (e.g., see 100 in FIGS. 1-2B). Some implementations may involve an actual test that assesses the user's breathing and general lung function.

Other implementations may incorporate breathing routines intended to activate the user's lungs before administering a test to determine the quality of the user's lung function. In still other implementations, the supplemental breathing test may actually be a form of training for the user's lungs, and may thereby serve as more of a supplemental breathing exercise that is designed to improve (e.g., condition) one or more specific respiratory characteristics of the user. For example, a user concerned with improving the length of time they can hold their breath may initiate a breath holding exercise while donning a user interface before entering a sleep session and/or after waking from a sleep session while still donning the user interface. In some implementations, the user may perform breath holding exercises at various times of the day unrelated to a sleep session, for example, as part of a training program which may include one or more exercises of which the intensity of the exercise (e.g., length or difficulty level) may vary at a pre-determined rate so as to achieve a particular goal.

Although various ones of the processes included in method 600 have been described as being performed in response to receiving an input from a user, this progression is in no way intended to limit the invention. For instance, the input received in operation 602 may actually be provided by the user in response to a prompt that is presented to the user. According to various examples, the respiratory therapy system may display a prompt on a HMI (e.g., see 150 of FIGS. 1-2B), send a prompt to a user's mobile device over a network, push a prompt to a smartwatch (e.g., see 260 of FIGS. 1-2B) over a network, play an audio signal that summarizes a prompt on a speaker of the respiratory therapy device, etc. The type of network used to deliver the prompt to the user may thereby also vary depending on the implementation. For instance, in some approaches the network is a WAN, e.g., such as the Internet. However, an illustrative list of other network types which may be used includes, but is not limited to, a LAN, a PSTN, a SAN, an internal telephone network, etc.

It follows that the input received in operation 602 may be an input that was initiated by the user themselves in some implementations. For instance, a user that wants to test their lung function may proactively provide an input to the respiratory therapy system that selects a desired supplemental breathing test to be performed. In one example, the input is received from the user following a sleep session, e.g., after the user wakes up. In another example, the input is received from the user prior to entering a sleep session. However, in other implementations the input may be received in response to an inquiry that was already sent to the user. According to yet another example, the respiratory therapy system sends a prompt to the user, encouraging them to test their lung function and see how much it has improved. This prompt may be sent to the user after a predetermined amount of therapy has been performed, in response to detecting an improvement in breathing during sleep sessions, etc.

Although not illustrated in FIG. 6A, instructions may be provided to the user in response to receiving the input in operation 602. In other words, instructions that correspond to the supplemental breathing test may be made available to the user. The user may thereby be prompted with details associated with the supplemental breathing test before actually administering the supplemental breathing test. This gives the user a better understanding of the process(es) involved with performing the supplemental breathing test, preferably such that the user is able to provide rich data samples which are ultimately able to achieve a more accurate understanding of the user's lung function.

The instructions may be provided to the user in a number of different ways, e.g., depending on the implementation. For instance, in some implementations, the instructions may be provided to the user by displaying textual and/or pictorial representations of the instructions on a display of a respiratory therapy device (e.g., see 150 of FIGS. 1-2B). In other implementations, the instructions may be provided to the user by playing an audio signal that summarizes the instructions on a speaker of the respiratory therapy device (e.g., see 222 of FIG. 1). In still other approaches, the instructions may be provided to the user by transmitting information to a mobile device (e.g., phone, tablet, etc.), a wearable device (e.g., smartwatch, smart glasses, etc.), etc., over a network.

Operation 604 further includes administering the supplemental breathing test. In other words, operation 604 includes adjusting parameters (e.g., operating parameters) of the respiratory therapy device, such that the supplemental breathing test may be run. Accordingly, the operating settings of one or more components in the respiratory therapy device may be changed according to the supplemental breathing test. For example, a motor in the respiratory therapy device may be instructed to operate (e.g., run) at one or more predetermined speeds to produce an airflow having a specific pressure supplied from the respiratory therapy device to the user via the conduit and user interface. This airflow thereby causes a predetermined air pressure, different from air pressures implemented during ordinary (e.g., standard) use of the respiratory therapy device, in an interior of the user interface. The predetermined air pressure corresponds to the supplemental breathing test for the user. In another example, one or more retractable partitions may be activated such that ambient air is at least partially prevented from entering and/or leaving an interior of the user interface and conduit, e.g., to test a user's lung capacity. For instance, the partitions may be extended to cover at least some of the vents in the user interface (e.g., see 372, 376 of FIGS. 3A-3B) such that the user experiences a simulated breathing environment. According to an example, which is in no way intended to limit the invention, a user who is hyperventilating may be at least partially restricted from inspiring ambient air.

It follows that each of the supplemental breathing tests that are supported by a given respiratory therapy system may correspond to predetermined operating settings of the components actually in the respiratory therapy system. These predetermined operating settings may be identified during a manufacture process of the respiratory therapy system, during one or more calibration processes performed between uses, downloaded and saved in memory during a software update, etc. Accordingly, operation 604 may include accessing a lookup table to determine the specific operating settings predetermined as corresponding to the specific supplemental breathing test being administered. One or more instructions may thereby be sent to various ones of the components in the respiratory therapy system such that the specific operating settings are implemented, and the supplemental breathing test can be administered.

Again, the manner in which the supplemental breathing test is administered depends on details associated with the specific type of breathing test. As noted above, the supplemental breathing test is intended to refer to any breathing related process that may be administered by a respiratory therapy system (e.g., see 100 in FIGS. 1-2B). Some implementations involve an actual test that assesses the user's breathing and general lung or upper airway function, while other implementations incorporate breathing routines intended to activate and/or train the user's lungs or upper airway. Accordingly, the process of actually administering the supplemental breathing test will also vary depending on the specific implementation. However, it should be noted that the user interface is preferably donned by the user during the supplemental breathing test. This allows for the respiratory therapy device to control the pressure, flow, humidity, etc., of the air that is provided to the user, and for the system to have an accurate understanding of how the user's respiratory system operates, e.g., as will be described in further detail below.

It is also preferred that the supplemental breathing test is not administered before the user is aware of what the supplemental breathing test involves. Accordingly, the test is administered following the user being provided with instructions. This gives the user an opportunity to determine the testing procedure and understand their role. In some implementations, the supplemental breathing test may actually be administered in parallel with the instructions being provided to the user. For instance, airflow is generated by the respiratory therapy device as a part of the supplemental breathing test, in parallel with the instructions being provided to the user. It is also preferred that the instructions remain available to the user during the entirety of the supplemental breathing test, e.g., such that they may be referenced as desired.

In some implementations, the supplemental breathing test involves generating airflow (e.g., air pressures, flow rates, humidity levels, etc.) that is noticeably different than airflow experienced by the user during standard (e.g., average) operation of the respiratory therapy device. Accordingly, it may be desirable to provide the user with the option of ending the supplemental breathing test at any time. This allows for the user to avoid injuries, experiencing anxiety, etc., associated with participating in the supplemental breathing test. The user may thereby be able to selectively provide an input that effectively acts as an emergency stop for the supplemental breathing test. For example, the user may be able to provide a specific input that indicates the airflow generated by the respiratory therapy device in accordance with a supplemental breathing test is uncomfortable (e.g., unbearable). Thus, the supplemental breathing test may immediately be ended in response to receiving a predetermined input from the user.

In some instances, the supplemental breathing test may be ended by returning the one or more components in the respiratory therapy device to standard operating conditions. In other implementations, the supplemental breathing test may be ended by cutting the power supply to one or more components in the respiratory therapy device. In still other implementations, the input received from the user may initiate a gradual reduction in the operating parameters of various components in the respiratory therapy device, e.g., to determine an upper limit based on the user's continued input.

It may be desirable to ensure the user is prepared for the supplemental breathing test to begin, before actually administering the test. Referring momentarily to FIG. 6B, an exemplary sub-method 630 of operations for ensuring the user is ready for the supplemental breathing test to begin, which may be performed based on at least some of the information received and/or determined in method 600. In some implementations the operations in sub-method 630 may be performed in the background by a same or different element or aspect of the system 100 (FIGS. 1-2B) as used to perform one or more steps of method 600. However, any one or more of the operations in sub-method 630 may be performed by any desired type of component (e.g., processor).

Looking to FIG. 6B, decision 632 includes determining whether an acknowledgement corresponding to a queued supplemental breathing test has been received from a user. As noted above, some supplemental breathing tests may involve airflows that are substantially different than airflow experienced by the user during standard (e.g., average) operation of the respiratory therapy device. Accordingly, the acknowledgement may effectively indicate that the user is aware of the supplemental breathing test and/or the details (e.g., generated airflows, testing instructions, etc.) associated therewith. It should also be noted that the acknowledgement may be received in any desired manner, e.g., over a wireless network, over a wired connection, etc.

In response to determining that an acknowledgement corresponding to a queued supplemental breathing test has not yet been received from the user, the flowchart is depicted as proceeding to operation 634. There, operation 634 includes waiting (e.g., honoring) a predetermined amount of time before returning to decision 632. The amount of time separating attempts at determining whether an acknowledgement has been received from the user may be predetermined according to the type of breathing test being administered, user preferences, a number of previous attempts that have been made, etc. It should also be noted that the performance of subsequent determinations may be based on other factors, e.g., such as whether an additional notification has been sent to the user.

It follows that decision 632 and operation 634 may be repeated in an iterative fashion any desired number of times. For instance, in some implementations these processes are repeated until an acknowledgement is actually received from the user (or the device is powered off), while in other implementations the flowchart may deny the supplemental breathing test in response to a predetermined amount of time having passed.

Returning to decision 632, the flowchart proceeds to operation 636 in response to determining that an acknowledgement corresponding to a queued supplemental breathing test has been received from the user. In other words, the supplemental breathing test is administered after the user confirms they are ready for it to begin, e.g. as a safety check. There, operation 636 includes actually administering the supplemental breathing test.

As noted above, the process of administering a supplemental breathing test may differ depending on the particular test. For instance, some supplemental breathing tests may involve a respiratory therapy device actively generating an airflow provided to a user interface donned by the user such that the user experiences a particular pressure, humidity, flow rate, etc., while attempting to breath into an interior of the user interface. Accordingly, in some implementations, the process of administering the supplemental breathing test involves sending one or more operating instructions to various components in the respiratory therapy device such that the respective components function in a desire manner and the supplemental breathing test may be administered.

However, other supplemental breathing tests may not involve positively generating an airflow at all. For example, a supplemental breathing test may limit the amount of oxygen that is available to the user while donning the user interface by at least partially blocking ambient air containing new oxygen from entering and/or exiting the user interface. In other words, administering some supplemental breathing tests may effectively convert the respiratory therapy system into a closed breathing apparatus that is able to counteract the onset of hyperventilation. This may be achieved by at least partially blocking ambient air containing new oxygen from entering and/or exiting the user interface, conduit, and respiratory therapy device such that the amount of oxygen available to the user is limited, e.g., as would be appreciated by one skilled in the art after reading the present description. In such situations, the motor, humidifier, etc., may intentionally be deactivated (e.g., instructed not to run).

An airflow is ultimately received from the user. In other words, air produced by the user while exhaling (i.e., exhaled air) during the course of the supplemental breathing test is received from the user to an interior of the user interface and the conduit. As noted above, the supplemental breathing test is preferably administered while the user interface is donned by the user. This allows for the respiratory therapy system to directly receive airflow generated by the user while they are breathing. Various sensors positioned throughout the respiratory therapy system (e.g., in the user interface, the conduit, etc.) may thereby collect information corresponding to the user generated airflow. For example, one or more flow sensors and/or pressure sensors may be positioned in an interior of the user interface, along an interior of the conduit, in the respiratory therapy device, etc., the one or more sensors being configured to collect information associated with air exhaled by the user during a supplemental breathing test. Accordingly, operation 606 further includes receiving airflow parameter data associated with the airflow received from the user.

As noted above, the airflow parameter data may be received from one or more sensors associated with the respiratory therapy device. Moreover, this airflow parameter data may be used to determine various details associated with the user's lung function. For instance, in some instances the airflow parameter data may be used to determine an approximate lung capacity (e.g., within 5% of the actual lung capacity) of the user. In other instances, the airflow parameter data may be used to determine improvements to specific lung functions of the user. According to an example, the airflow parameter data received from the user during the supplemental breathing test may be compared against airflow parameter data received during previous supplemental breathing tests, sleep sessions, etc., to determine specific improvements to the user's lung function, e.g., as will be described in further detail below.

The one or more sensors from which the airflow parameter data is received may be coupled to various portions of the respiratory therapy device, positioned in a same room as the respiratory therapy device, configured to communicate with one or more components in the respiratory therapy device, etc. According to an example, which is in no way intended to limit the invention, acoustic-based airflow parameter data may be received from one or more transducers that are configured to convert sound waves into electrical signals. These transducers may be positioned on an inner surface of the user interface, along the conduit, adjacent to a motor of the respiratory therapy device, etc., such that sound waves associated with a user breathing into the user interface and/or airflow being generated may be identified in real-time.

As noted above, various types of airflow parameter data received from at least some sensors may be stored in memory over time and even analyzed to develop an accurate understanding of the user's lung function during various scenarios. For instance, one or more breathing samples may be received from the sensors while intentionally operating the respiratory therapy device in certain settings. According to an example, a user may be prompted to don the user interface and perform a spirometry test by taking a deep breath and exhaling as much air from their lungs as possible. The user may be prompted to exhale air against a variable pressure in some implementations, e.g., to simulate a desired breathing environment as part of a supplemental breathing test. In other implementations, the user may be prompted to perform the spirometry test against a fixed pressure of about 1 standard atmosphere (e.g., the mean sea-level atmospheric pressure on Earth).

Sensors are thereby able to collect various types of airflow parameter data (e.g., acoustic data, flow rate data, temperature data, pressure data, humidity data, etc.) associated with the airflow generated while the user is exhaling into the interior of the user interface. According to some preferred implementations, the airflow data received in operation 606 may include flow data and pressure data.

It should also be noted that the airflow parameter data received in operation 606 preferably includes useable data for the airflow received from the user as a result of their breathing (e.g., general lung activity). In other words, as much information (e.g., data) as possible is collected during the supplemental breathing test, preferably such that rich data samples associated with the user's lung function may be collected by the various sensors. According to an example, which is in no way intended to limit the invention, a sampling rate of the various sensors may be increased to a rate that is sufficient to collect rich data associated with the user's lung function, e.g., as would be appreciated by one skilled in the art after reading the present description.

Each discrete operating condition of the components in the respiratory therapy device is also preferably maintained long enough for rich data samples associated with the given supplemental breathing test to be collected by the various sensors. According to an example, which again is in no way intended to limit the invention, a motor in the respiratory therapy device may produce a specific number of revolutions per minute for a period of time sufficient for sensors in and/or around the respiratory therapy device to collect rich data associated with the user's lung function (e.g., breathing performance) at a specific air pressure, e.g., as would be appreciated by one skilled in the art after reading the present description.

With continued reference to FIG. 6A, operation 608 includes characterizing the lung function of the user. This characterization of the user's lung function is preferably based, at least in part, on the airflow parameter data received from the various sensors in operation 606. As noted above, the airflow parameter data further corresponds to the airflow received from the user during the breathing test. Accordingly, the characterization of the user's lung function is determined based on how the user performs during the supplemental breathing test.

However, this characterization may also consider past respiratory performance. For example, process of characterizing the user's lung function may incorporate airflow parameter data received from the various sensors during past sleep sessions, previous breathing tests, etc. Physical attributes of the user may also be taken into consideration. For example, the age, size, etc. of a user may impact the abilities of their respiratory system, and therefore may be taken into consideration while characterizing that user's lung function.

With respect to the present description, a user's lung function may be characterized differently depending on the implementation. For instance, in some situations a particular aspect about a user's lung function may be characterized by simply averaging a number of airflow parameter data readings to determine a mean value. According to an example, the amount (e.g., volume) of air exhaled by a user during a lung capacity test may be averaged against results of all past lung capacity tests for the same user to determine an average value. In other situations, aspects of a user's lung function may be characterized by using any physiological, mathematical, biological, etc. relationships that would be apparent to one skilled in the art after reading the present description.

It follows that the specific aspects of a user's lung function characterized in operation 608 may vary depending on the implementation (e.g., see FIG. 6D below). Although not shown, one or more values that represent at least a portion of the lung function of the user may further be output (e.g., to the user). After characterizing the lung function of the user, various information associated with the user's specific lung function may be known to at least a certain degree of certainty. Again, the accuracy of the airflow parameter data received has an impact on how well the user's lung function is understood. Accordingly, it is preferred that the sensors are able to collect rich data during the supplemental breathing test, e.g., as would be appreciated by one skilled in the art after reading the present description.

Again, various information associated with the user's specific lung function may be determined in operation 608. For instance, in some implementations the one or more values output to the user include an amount (e.g., volume) and speed (e.g., flow) of air that can be inhaled and/or exhaled by the user in a single breath of ambient air. In other implementations, the one or more values output may indicate (e.g., determine, predict, identify a best recorded result, etc.) an amount of time the user can hold a single breath of ambient air. In still other implementations, the one or more values indicate a relative fitness score of the user. The fitness score may further identify the relative breath strength and related respiratory metrics for the user. In other words, the one or more values may indicate a level of fatigue experienced by the user.

It should also be noted that the one or more values may be output to a number of different destinations depending on the implementation. For instance, in some implementations the values may be output to a display on the respiratory therapy device (e.g., see 150 of FIGS. 1-2B), a mobile device, a piece of wearable technology (e.g., see 260 of FIGS. 1-2B), etc., for the user to at least visually access. In other implementations, the values may additionally or alternatively be sent to a personal trainer and/or medical professional for analysis, saved in local memory for additional processing, used to dynamically update operating conditions of the respiratory therapy device, etc. For example, the user's lung function may be used to determine whether the operating speed of the motor and/or the settings of the humidifier should be adjusted to improve the quality of sleep experienced by the user donning the user interface during subsequent sleep sessions.

From operation 608, the flowchart of FIG. 6A is illustrated as ending. However, it should be noted that although method 600 may end in response to performing operation 608, any one or more of the processes included in method 600 may be repeated in order to administer additional breathing tests. In other words, any one or more of the processes included in method 600 may be repeated for subsequent inputs received from users wishing to experience supplemental breathing tests.

It follows that the various processes included in method 600 are desirably able to determine lung function of a respiratory therapy device user, and track improvements to the lung function over time. As noted above, while the benefits of using the various respiratory therapy systems included herein are substantial, these benefits may not always be readily apparent to an individual that uses a respiratory therapy system. As a result, some users may not fully appreciate the benefits associated with continued use of a respiratory therapy system, and are less likely to continue use. Method 600 thereby provides users the opportunity to understand the various benefits that are achieved as a result of utilizing respiratory therapy systems. Again, this acts as a positive feedback loop, encouraging users to continue using respiratory therapy systems during sleep sessions, and ultimately improving user experience.

It follows that airflow parameter data received during use of the respiratory therapy device may constantly, periodically, occasionally, etc., be monitored to maintain an accurate understanding of users'respective lung function. While not specifically depicted in FIG. 6A, method 600 may thereby monitor breathing performance of a user in the background and automatically identify changes to the user's lung function. This allows for performance of the respiratory therapy device to be adjusted in real time such that the quality of sleep experienced by the user during a sleep session is optimized.

FIG. 6C illustrates an exemplary sub-method 640 of operations for monitoring a user's lung function, which may be performed based on at least some of the information received and/or determined in method 600. In some implementations the operations in sub-method 640 may be performed in the background by a same or different element or aspect of the system 100 (FIGS. 1-2B) as used to perform one or more steps of method 600. However, any one or more of the operations in sub-method 640 may be performed by any desired type of component (e.g., processor).

By monitoring the lung function of a user while using a respiratory therapy device, changes may be identified during use over time. These changes in lung function may be accounted for by selectively adjusting operating settings of the various components in the respiratory therapy device, e.g., such that the user is able to maintain a desirable quality of sleep across various sleep sessions. Lung function may also be used to determine additional information associated with a given user. For example, breathing performance of a user may indicate the onset of one or more medical conditions (e.g., medical reactions). Thus, by monitoring the user's lung function, these medical conditions may be identified and counteracted, e.g., as will soon become apparent.

Looking to FIG. 6C, operation 642 includes monitoring breathing performance of the user. In some implementations, various sensors throughout a respiratory therapy system may be instructed to continue collecting data associated with an individual's use of the system. For example, airflow parameter data received while a user is donning the user interface (e.g., prior to a sleep session, during a sleep session, during subsequent sleep sessions, after a sleep session, etc.) may be collected and evaluated to determine any notable changes in the user's breathing performance and corresponding lung function.

As noted above, this airflow parameter data may also be used to identify the onset of one or more medical conditions (e.g., medical reactions). Accordingly, decision 644 includes determining whether the user is potentially experiencing the onset of a medical condition. In other words, a determination is made as to whether the user is beginning to experience at least a possible onset of a medical condition. The type and/or amount of airflow parameter data available may determine the types of medical conditions method 640 is able to identify. However, depending on the implementation, decision 644 may be able to identify the potential onset of an asthma attack, an anxiety attack, a respiratory infection, etc., or any other type of respiratory-related medical condition.

Again, this determination may be made using the airflow parameter data that is received while monitoring the user's breathing performance. For instance, this airflow parameter data may be compared against previous data readings (e.g., standard data readings) stored in memory, predetermined ranges that may be used to identify unusual respiratory performance, supplemental information supplied by the user (e.g., medical test results, doctor instructions, prescription side effects, etc.), etc. Thus, by monitoring the user's breathing performance, method 640 is able to identify breathing performance that indicates the onset of these medical conditions. Additional action may thereby be taken in order to counteract the identified medical conditions.

In response to determining that the user is not experiencing the onset of a medical condition, method 640 returns to operation 642 from decision 644. Accordingly, the breathing performance of the user may continue to be monitored (e.g., during a sleep session) before returning to decision 644.

However, in response to determining that the user is potentially experiencing the onset of a medical condition, method 640 proceeds to operation 646. There, operation 646 includes adjusting performance of one or more components in the respiratory therapy device to counteract the medical condition. In other words, by adjusting the operating parameters of various components in the respiratory therapy device (e.g., the motor, humidifier, etc.), the method 640 is desirably able to combat the onset, severity, extent, etc. of the medical condition.

According to an example, which is in no way intended to limit the invention, monitoring the user's breathing performance after waking from a sleep session may indicate that the user is beginning to experience hyperventilation. Accordingly, the respiratory therapy system may intentionally close one or more vents (e.g., see 372 of FIGS. 3A-3B) and/or a portion of the conduit (e.g., see 140 of FIGS. 1-2B), thereby limiting an amount of oxygen available to the user while donning the user interface. This may effectively convert the user interface into breathing apparatus that is able to counteract the onset of hyperventilation by at least partially blocking the supply of oxygen to the user. In other words, by blocking ambient air containing new oxygen from entering the user interface, conduit, and/or respiratory therapy device, the increasingly carbon dioxide rich air trapped in the user interface counteracts the respiratory alkalosis experienced by the user, and avoids the hyperventilation from progressing.

It follows that method 640 is able to identify breathing performance that indicates the onset of these medical conditions, and take preemptive steps to counteract the progression of such medical conditions. As a result, method 640 is desirably able to maintain an improved user experience and quality of sleep for the user.

According to some implementations of the present disclosure, monitoring the user's breathing performance before entering a sleep session may indicate that the user is stressed. For instance, the user's breathing performance may indicate they are anxious and/or experiencing a panic attack. In order to calm the user down, making it easier for them to fall asleep and experience an improved quality of sleep, the respiratory therapy system may actually encourage the user to inhale and exhale at a predetermined cadence. In some implementations, this may be achieved by creating positive and negative air pressures that encourage the user to inhale and exhale as desired. This controlled breathing may help reduce the user's stress and actually avoid panic attacks from developing or worsening. The respiratory therapy system may also be able to implement guided meditation by encouraging users to breath at a particular pace and/or intensity, e.g., as described above.

It follows that method 640 is also able to identify breathing performance that indicates a particular mental state of the user, and take tailor performance of the respiratory therapy system to address that mental state and ensure an improved quality of sleep during subsequent sleep sessions. As a result, method 640 is desirably able to maintain an improved user experience and quality of sleep for the user.

As noted above, the process of characterizing a user's lung function is determined based on how the user performs during the supplemental breathing test. However, the specific aspects of a user's lung function that are actually characterized may vary depending on the implementation. FIG. 6D depicts an exemplary sub-method 650 of operations for characterizing the lung function of a user, which may be performed based on at least some of the information received and/or determined in method 600. In some implementations the operations in sub-method 650 may be performed in the background by a same or different element or aspect of the system 100 (FIGS. 1-2B) as used to perform one or more steps of method 600. However, any one or more of the operations in sub-method 650 may be performed by any desired type of component (e.g., processor).

As shown, method 650 includes determining an identity of the user. See operation 652. In preferred implementations, the airflow parameter data collected while the user conducted the supplemental breathing test is used to identify the user. In other words, the sleep data gathered is used to identify the user or at least determine certain identifying information about the user (e.g., their age, size, etc.).

By identifying the user to some extent, performance of the respiratory therapy device may be tailored to best suit the known aspects of the user. Moreover, the airflow parameter data collected during the supplemental breathing test may be compared against previously received data. Accordingly, operation 654 includes comparing the first airflow parameter data with known lung function data associated with the identified user. This known lung function data may be stored in memory.

Furthermore, decision 656 includes determining if the lung function of the user has improved. As previously noted, the continued use of the various respiratory therapy devices included herein desirably improves the lung function of users. However, these improvements are not always readily apparent to the users. Thus, by determining specific improvements to lung function of a user compared to previous data readings, the user may be significantly encouraged to continue using a respiratory therapy device.

In response to determining that the lung function of the user has improved (at least to some extent), method proceeds from decision 656 to operation 658. There, operation 658 includes outputting a report that summarizes the determined improvements to the user's lung function. The report also preferably correlates these determined improvements with the amount of time spent using the respiratory therapy device (e.g., during sleep sessions). In other words, improvements to a user's lung function are preferably reported to the user in such a way that the user is made aware of the fact that their continued use of the respiratory therapy device at least contributed to the realized improvements. This may desirably encourage the user to continue using the respiratory therapy device during subsequent sleep sessions, thereby resulting in an improved user experience.

In addition to outputting a report that summarizes the determined improvements to the user's lung function, operation 660 includes updating standard operating values assigned to one or more of the parameters of the respiratory therapy device. In other words, operation 660 includes updating the respiratory therapy device to operate differently during standard use based on the improvements that have been achieved to the user's lung function. These updated standard operating values desirably allow for the user to experience an improved quality of sleep during subsequent sleep sessions. The updated standard operating values even allow for the user's lung function to continue improving. Thus, the updated standard operating values implemented may be tailored to accentuate certain aspects of the user's lung function and/or deemphasize aspects of the user's lung function. For example, a motor in the respiratory therapy device may be instructed to operate (e.g., run) at an updated standard operating speed during subsequent sleep sessions to produce an airflow having a specific pressure supplied from the respiratory therapy device to the user to continue improving the user's lung function.

From operation 660, the flowchart proceeds to operation 664, whereby method 650 may end. However, it should be noted that although method 650 may end upon reaching operation 664, any one or more of the processes included in method 650 may be repeated in order to characterize the lung function of additional users.

Returning to decision 656, method 650 proceeds directly to operation 662 in response to determining that the lung function of the user has not improved. In other words, method 650 proceeds to operation 662 in response to determining that the lung function of the user has remained the same or decreased (e.g., worsened). There, operation 662 includes suggesting one or more changes to the user in an attempt to improve their lung function. For instance, operation 662 may include suggesting changes to the standard operating values assigned to the parameters of the respiratory therapy device. As noted above, changes to these standard operating values may impact how the respiratory therapy device performs.

These changes may be tailored in an attempt to improve the lung function of the user during subsequent sleep sessions. For instance, past respiratory performance of the user may be evaluated and compared against the standard operating values that were implemented. Correlations between standard operating values assigned to the parameters of the respiratory therapy device and the user's lung function may be made using various mathematical relationships, statistical modeling, deep-learning processes, etc., as would be appreciated by one skilled in the art after reading the present description. Moreover, these determined (e.g., learned) correlations may be implemented during subsequent use of the respiratory therapy device, thereby improving the user's sleep quality and overall enjoyment.

While attempts to counteract stagnant or decreasing lung function may be made at the respiratory therapy device itself, additional action may be warranted depending on the situation. For instance, in some instances, lung function of the user may decrease at a rapid rate and/or indicate the possible onset of a medical disorder. Accordingly, in some implementations, method 650 includes actively prompting the user to schedule an appointment with a medical professional in response to determining that the lung function of the user has decreased.

It should also be noted that while various ones of the implementations herein have been described in the context of determining the lung function of a user, this is in no way intended to be limiting. According to an example, any one of the implementations herein may be similarly applied to monitor, test, and evaluate lung stiffness of a user. Lung stiffness may be monitored to optimize a rehabilitation program. For instance, lung stiffness is impacted by lung injury, e.g., such as broken rib(s), tissue damage, etc. In some implementations a model of the damped natural frequency of the lung is formed to extract the lung stiffness from measurements of oscillations in the respiratory flow signals.

According to another example, the exponential decay of a respiratory flow signal during expiration to a model of the lungs including resistance and compliance may be utilized. Input may be received from a source, e.g., such as a clinician, to define a type of injury experienced by the user. The input may also indicate duration of rehabilitation, demographic info (e.g., such as age, BMI, etc.), medical notes, etc., of a patient or user of the respiratory therapy system. Operating settings of the respiratory therapy system may thereby be set to accommodate the particular medical state of the user. Over a period of training or rehabilitation, the user of the respiratory therapy system may be prompted to gradually adjust respiratory parameters as their recovery progresses. For instance, the respiratory therapy system may slowly guide the user to transition from a faster respiratory rate and smaller inspired volume of air, to a slower respiratory rate and a larger volume of inspired air. This may desirably allow for the user to maintain a particular ventilation rate while also gradually increasing the expansion of their lungs with continued use of the respiratory therapy system.

One or more elements or aspects or steps, or any portion(s) thereof, from one or more of any of claims 1 to 66 below can be combined with one or more elements or aspects or steps, or any portion(s) thereof, from one or more of any of the other claims 1 to 66 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1-35. (canceled)

36. A respiratory therapy system comprising:
a respiratory therapy device;
a conduit;
a user interface, the user interface being coupled, via the conduit, to the respiratory therapy device;
a memory device having stored thereon machine-readable instructions; and
a control system including one or more processors configured to execute the machine-readable instructions to:
in response to receiving an input from the user selecting a supplemental breathing test, administering the supplemental breathing test by adjusting parameters of the respiratory therapy device;
receiving first airflow parameter data associated with a first airflow generated by the user into an interior of the user interface while donning the user interface; and
characterizing the lung function of the user based at least in part on the first airflow parameter data.

37. The respiratory therapy system of claim 36, wherein administering the supplemental breathing test includes generating a second airflow from the respiratory therapy device to the conduit by operating a motor of the respiratory therapy device at one or more discrete operating speeds.

38. The respiratory therapy system of claim 37, wherein the received first airflow parameter data includes useable data collected at each of the one or more discrete operating speeds of the motor.

39. The respiratory therapy system of claim 37, wherein the second airflow causes a predetermined air pressure in the interior of the user interface, the predetermined air pressure corresponding to the supplemental breathing test.

40. The respiratory therapy system of claim 39, wherein the second airflow is generated in response to receiving an acknowledgement from the user.

41. The respiratory therapy system of claim 37 wherein administering the supplemental breathing test includes providing instructions to the user that correspond to the supplemental breathing test, wherein the second airflow is generated in parallel with the instructions provided to the user.

42. The respiratory therapy system of claim 41, wherein providing the instructions to the user includes displaying the instructions on a display of the respiratory therapy device.

43. The respiratory therapy system of claim 42, wherein providing the instructions to the user includes playing an audio signal that summarizes the instructions on a speaker of the respiratory therapy device.

44. The respiratory therapy system of claim 36, wherein the one or more processors are further configured to execute the machine-readable instructions to:
in response to receiving a second input from the user, immediately ending the supplemental breathing test; and
returning the parameters of the respiratory therapy device to their standard operating values.

45. The respiratory therapy system of claim 44, wherein the second input indicates that the second airflow is uncomfortable to the user.

46. The respiratory therapy system of claim claims 36, wherein the supplemental breathing test is a spirometry test.

47. The respiratory therapy system of claim 36, wherein the one or more processors are further configured to execute the machine-readable instructions to:
monitoring breathing performance of the user;
identifying breathing performance that indicates an onset of a medical condition; and
adjusting the parameters of the respiratory therapy device to counteract the medical condition.

48. The respiratory therapy system of claim 47, wherein the medical condition is an asthma attack.

49. The respiratory therapy system of claim 47, wherein the medical condition is an anxiety attack.

50. The respiratory therapy system of claim 49, wherein the breathing performance is associated with hyperventilation, and wherein adjusting performance of the respiratory therapy device to counteract the medical condition includes: limiting an amount of oxygen available to the user while donning the user interface, by at least partially blocking ambient air from entering the user interface.

51-52. (canceled)

53. The respiratory therapy system of claim 36, wherein characterizing the lung function of the user includes:
determining an identify of the user based at least in part on the first airflow parameter data;
comparing the first airflow parameter data with known lung function data associated with the identified user; and
determining if the lung function of the user has improved.

54. The respiratory therapy system of claim 53, wherein characterizing the lung function of the user further includes:
in response to determining that the lung function of the user has improved, updating standard operating values assigned to one or more of the parameters of the respiratory therapy device, the updated standard operating values being configured to improve a quality of sleep experienced by the user during subsequent sleep sessions.

55. (canceled)

56. The respiratory therapy system of claim 53, wherein characterizing the lung function of the user further includes:
in response to determining that the lung function of the user has decreased, suggesting one or more changes to the standard operating values assigned to one or more of the parameters of the respiratory therapy device, the changes being configured to improve the lung function of the user during subsequent sleep sessions.

57. (canceled)

58. The respiratory therapy system of claim 36, wherein the one or more processors are further configured to execute the machine-readable instructions to:
outputting one or more values that represent at least a portion of the lung function of the user, the one or more values selected from the group consisting of: (i) an amount and/or a speed of air that can be inhaled and exhaled by the user in a single breath of ambient air, (ii) an amount of time the user can hold a single breath of ambient air, (iii) an amount of time the user can hold a single breath of ambient air, (iv) a fitness score of the user.

59.-64. (canceled)

65. The respiratory therapy system of claim 36, wherein the first airflow parameter data includes one or more airflow parameters selected from the group consisting of: acoustic data, flow data, and pressure data.

66. (canceled)
